(19) United States
(12) Patent Application Publication
(10) Pub. No.: US 2008/0044387 A1
(43) Pub. Date: Feb. 21, 2008

(54) ARTIFICIAL NICHES FOR ENHANCEMENT OF REGENERATIVE CAPACITY OF STEM CELLS IN AGED AND PATHOLOGICAL ENVIRONMENTS

(75) Inventors: Irina M. CONBOY, El Sobrante, CA (US); Maria Elena Juan PARDO, Berkeley, CA (US); Morgan CARLSON, Albany, CA (US)

Correspondence Address: MORGAN, LEWIS & BOCKIUS, LLP, ONE MARKET SPEAR STREET TOWER, SAN FRANCISCO, CA, 94105, US

(73) Assignee: THE REGENTS OF THE UNIVERSITY OF CALIFORNIA, Oakland, CA (US)

(21) Appl. No.: 11/688,794

(22) Filed: Mar. 20, 2007

Related U.S. Application Data
(60) Provisional application No. 60/785,564, filed on Mar. 24, 2006.

(51) Int. Cl. A61K 38/00; A61K 48/00; A61P 43/00

(52) U.S. Cl. 424/93.3; 514/12

(57) ABSTRACT

Artificial niches which provide growth, differentiation, and/or anti-aging environments for stem cells and progenitor cells are described.

### REFERENCES

This application claims benefit under 35 U.S.C. §119(c) of U.S. Ser. No. 60/245,840 filed 60/785,564 filed Mar. 24, 2006 which is hereby expressly incorporated by reference in its entirety.

### BACKGROUND OF THE INVENTION

Skeletal muscle is maintained and repaired by endogenous stem cells, called satellite cells, which constitute all the regenerative potential in this organ (Zammit et al., Exp. Cell Res., 281(1): 39-49 (2002)), (Sherwood et al., Cell, 119(4):543-554 (2004)). Satellite cells reside in direct contact with the differentiated, multinucleated muscle cells (myofibers or myotubes), under the basal lamina. Two to five percent of all muscle nuclei are in satellite cells in the adult muscle (Morgan et al., Int. J. Biochem. Cell Biol., 35(8): 1151-1156 (2003)). In resting adult muscle, 99.9% of satellite cells are mitotically quiescent until muscle injury activates satellite cells to proliferate and differentiate along myogenic lineage into the myogenic progenitor cells, which progress to become fusion competent myoblasts (Morgan et al., Int. J. Biochem. Cell Biol., 35(8):1151 - 1156 (2003)). Myoblasts are still capable of division but can also fuse to form new multinucleated myofibers. This coordinated cell-fate determination, which consists of cell expansion followed by differentiation, serves to repair or replace the damaged muscle (Morgan et al., Int. J. Biochem. Cell Biol., 35(8):1151-1156 (2003)).

Muscle regeneration is a complex process of tissue remodeling that involves myogenesis, re-enervation, re-vascularization and is regulated by an intricate network of biochemical pathways: including those initiated by inflammatory cytokines, growth factors, integrins, and the evolutionarily-conserved Notch, Wnt, and Shh signaling pathways (Conboy et al. Dev. Cell, 3 (3):397-409 (2002)). (Husmann et al., Cytokine Growth Factor Rev., 7(3): 249-258 (1996)), (Pola et al., Circulation, 108 (4), 479-485 (2003) (Seale et al., Cell Cycle, 2(5):418-419 (2003)), (Taverna et al., J. Cell Biol., 143(3):849-859 (1998)), (Tidball, Am J Physiol Regul Integr Comp Physiol, 288(2):345-53 (2005)), (Yang et al., J. Cell Biol., 135(3):829-835 (1996)). Injury promotes the release of growth factors that bind to extracellular matrix (ECM) proteins, such as proteoheparan sulfates (Husmann et al., Cytokine Growth Factor Rev., 7(3): 249-258 (1996)). During later stages of regeneration, interactions between the remodeled ECM and cell-surface integrin receptors play a key role in the adhesion and spreading of newly-generated myoblasts, thus the organization of the regenerated muscle fibers (Disatnik et al., J. Cell Sci., 115(10):2151-2163 (2002)). (Disatnik et al., J. Biol. Chem., 274,(45):32486-32492 (1999)), (Zaidel-Bar et al., Biochem. Soc. Trans., 32 (Pt3):416-420 (2004)). Among the best characterized growth factors, the most important in muscle repair are FGF-2, IGF-1, TGF-β and GDF-8 (myostatin). FGF-2 promotes proliferation of myogenic progenitor cells and delays their differentiation, in part by inhibiting the expression of myogenic regulatory factors (Maley et al., Exp. Cell Res., 211(1):99-107(1994)), (Miller et al., Am. J. Physiol Cell Physiol, 278(1):C174-C181 (2000)). IGF-1 promotes myogenic differentiation (Florini et al., Endocr. Rev, 17(5):481-517 (1996)) and is a key determinant of muscle mass, as it enhances protein synthesis in differentiated myofibers (Bodine et al., Nat. Cell Biol., 3(11):1014-1019 (2001)), (Latres et al., J. Biol. Chem., 280(4):2737-2744 (2005)). At the same time, IGF-1 down-regulates protein degradation in muscle cells (Sandri et al., Cell, 117(3):399-412 (2004)). (Stitt et al., Mol. Cell, 14(3):395-403 (2004)) and has anti-apoptotic effects (Downward, Semin. Cell Dev. Biol., 15(2):177-182 (2004)). Also. IGF-1 reduces age-related muscle atrophy and attenuates experimentally-induced muscle wasting (Chakravarthy et al., Mech. Ageing Dev., 122(12):1303-1320 (2001)), (Shavlakadze et al., Neuromuscl. Disord., 15(2):139-146(2005)). TGF-β and the muscle-specific TGF-β family member, myostatin, are on the opposite end of the proliferative spectrum. These factors inhibit proliferation of myogenic progenitor cells during both embryonic development and adult muscle regeneration (McCroskery et al., J. Cell Biol., 162, (6):1135-1147 (2003)), (MePherron et al., Nature, 387(6628):83-90 (1997)), (Moustakas et al., Immunol. Lett., 82(1-2):85-91(2002)), (Thomas et al., J. Biol. Chem., 275(51),40235-40243 (2000)), (Zimmers et al., Science, 296(5572):1486-1488 (2002)). Myostatin mRNA has been shown in vivo, to progressively accumulate during muscle repair (Armand et al., Dev. Dyn., 227(2):256-265 (2003)), while the mRNA levels of its inhibitor, follistatin, was shown to be present in the mono-nucleated muscle cells located near the injury site and in newly formed myofibers (Armand, et al., Dev. Dyn., 227(2):256-265 (2003)). This well-studied interplay of growth factors in regenerating muscle serves to restore cellular homeostasis during injury repair (Husmann et al., Cytokine Growth Factor Rev., 7(3): 249-258 (1996)).

In stark contrast to young animals, aged organisms produce very few myoblasts in response to muscle injury, and thus not enough cells are available to form new myofibers (Bockhold et al, Muscle Nerve, 21(2):173-183 (1998)), (Conboy et al., Science, 302(5650):1575-1577 (2003)). (Schultz et al. Mech. Ageing Dev., 20(4):377-383 (1982)). Decline in the generation of myoblasts in aged muscle has been proven not to be caused by a physical loss of satellite cells related to ageing (Conboy et al., Science, 302(5650):1575-1577 (2003)), but rather by a failure in their ability to become activated and proliferate in response to injury. Remarkably, the intrinsic satellite cell regenerative potential is not irreversibly lost with age, but rather is simply not triggered in old muscle due to extrinsic systemic factors (Conboy et al., Nature, 433(7027):760-764 (2005)). Our most recent data strongly suggest that it is not simply the lack of positive factors that cause the diminished satellite cell regenerative potential in aged organs, but that aged circulation has, in fact an inhibitory component that prevents tissue repair. Thus, the therapeutic value of stem cells becomes significantly diminished, unless the inhibitory components of aged organs are understood and their effects are countered.

It is of great interest to the scientific community to be able to control regeneration in chronically degenerating or aged organs either by in-situ activation of endogenous stem cells or by stem cell transplantation. Satellite cells have often been viewed as a promising source of regenerative reserve in transplantation studies. These cells are numerous in adult, readily available and relatively easily harvested; they rapidly expand in culture and their progeny myogenic progenitor cells also proliferate and are able to differentiate into new muscle tissue in vivo and in vitro (Morgan et al., Int. J. Biochem. Cell Biol., 35(84):1151-1156 (2003)). However, despite the decades of attempts using electroporation and other techniques, there is no known cell transplantation-based cure for repair of aged or pathologically degenerating muscle (Partridgye, Acta Neurol. Belg. 104(4):141-147 (2004)). Notably, taking into account the dependence of the satellite cells' regenerative potential on the extrinsic environment described above, the ability of transplanted cells to efficiently repair muscle is likely to be inhibited in the aged environment of a degenerating organ, even if the transplantation itself was successful.

This invention described below addresses these needs, as well as others.

### BRIEF DESCRIPTION OF THE DRAWINGS

FIG. 1. Geometric control of terminal myogenic differentiation in growth media. Myogenic progenitor cells have been plated at 50% confluency in chamber slides in growth media (GM)
(A): GM with unmodified ECM (GM) and GM with DF-modified ECM (GM+DF).
(B): Quantification of proliferating cells (P), differentiated cells with less than 2 nuclei (D1=early stage of differentiation) and differentiated cells with more than 2 nuclei (D2=later stage of differentiation). On unmodified ECM, there is a significantly higher percentage of proliferative cells versus differentiated cells. Looking at cells grown on DF-modified ECM, we see higher numbers of differentiated cells, but most cells do not form multinucleated myotubes.
(C): The boundary between unmodified ECM substrate (outside) and DF-modified ECM (inside). Geometric boundary between DF-modified and unmodified ECM substrate was created as described in Methods. Cells were uniformly seeded throughout the ECM area, cultured for 48 h and fixed. Immunofluorescence was performed with the indicated antibodies: α-BrdU (red), α-eMHC (green) and Hoechst (blue) was used to label all nuclei. Proliferation (incorporation of BrdU) is observed in GM with unmodified ECM and inhibited proliferation and differentiation (expression of eMHC) is observed in GM with DF-modified ECM. Similar results have been obtained in at least three independent experiments.

FIG. 2. Geometric delay of myotube formation in differentiation media by locally embedded growth factors. Myogenic progenitor cells have been plated at 50% confluency in chamber slides in differentiation media (DM). Cells were cultured for 48 h and fixed. Immunofluorescence was performed with the indicated antibodies: α-BrdU (red), α-eMHC (green) and Hoechst (blue) was used to label all nuclei.
(A): DM with unmodified ECM (DM) and DM with GF-modified ECM (DM+GF). There is a clear difference in the fate of cells cultured in DM on unmodified ECM (terminally differentiated, multinucleated myotubes) versus on GF-modified ECM (higher numbers of proliferative cells and smaller myotubes).
(B): Quantification of proliferating cells (P), early stage-differentiated cells with less than 2 nuclei (D1) and later stage-differentiated cells with more than 2 nuclei (D2). Cells cultured on unmodified ECM in DM show low percentage of proliferating cells and high percentage of differentiated cells. Alternately, when cultured on GF-modified ECM, cells show higher percentage of proliferating cells and lower numbers of differentiated cells.
(C): The boundary between unmodified ECM substrate and DF-modified ECM is shown (DM+GF interface). Magnified photographs (20×) of cells seeded on unmodified ECM (outside) and on GF-modified ECM (inside) areas of the culture plate are also shown. Cells were originally seeded at uniform confluency; however, as expected, cells adherent to the GF-modified ECM proliferated at a higher rate, resulting in a higher number of cells as compared to those adherent to control ECM.
Similar results have been obtained in at least three independent experiments.

FIG. 3. Geometric control of proliferation or terminal differentiation in neutral media. Myogenic progenitor cells have been plated at 50% confluence in chamber slides in differentiation media (NM) for 48 h. Immunofluorescence was performed with the indicated antibodies: α-BrdU (red), α-eMHC (green) and Hoechst (blue) was used to label all nuclei.
(A): Cells cultured in NM on unmodified ECM substrate (NM) show no distinct tendency towards proliferation or differentiation. When cultured on GF-modified ECM (NM+GF) cells proliferate (BrdU incorporation) without any tendency to form myotubes and differentiate, i.e. eMHC⁺. Conversely, when exposed to DF-modified ECM (NM+DF), cells terminally differentiate (eMHC⁺) and form myotubes while proliferation is reduced.
(B): Quantification of proliferating cells (P), early stage-differentiated cells with less than 2 nuclei (D1) and later stage-differentiated cells with more than 2 nuclei (D2). When cultured in NM on unmodified ECM, cells infrequently differentiate and have slow proliferation rate. However, when cells are plated on GF-modified ECM (NM+GF) there is a much larger percentage of proliferating cells; and when they are plated on DF-modified ECM (NM+DF) there are higher percentages of not only eMHC⁺ differentiated cells but also yield higher percentages of multinucleated myotubes (D2).
(C): Boundary between GF-modified ECM and unmodified ECM. There are higher numbers of proliferating cells on GF-modified ECM (inside) than on the unmodified ECM (outside).
Similar results have been obtained in at least three independent experiments.

FIG. 4. Delay of myogenic differentiation by locally embedded growth factors into ECM under high cell density (80% contluency). Myogenic progenitor cells have been plated at 80% confluency in chamber slides in differentiation media (DM) for 36 h. Immunofluorescence was performed after fixation with the indicated antibodies: α-Ki67 (red), α-eMHC (green) and Hoechst (blue) was used to label all nuclei. Specified in Table 1 growth factors (GF) were embedded into ECM in geometric fashion as shown in FIG. 6.

Consistent with the control differentiation medium (DM) shown in FIG. 2A-DM, cells outside the geometric boundary (outside) form eMHC positive robust myotubes and do not proliferate. Myogenic cell differentiation is diminished as indicated by the lower number of nuclei per myotubes and some Ki67⁺ proliferating cells persist inside the geometric boundary (inside). Thus, GF embedded in the ECM are capable of diminishing differentiation even when cell numbers increase, but differentiation seems inescapable despite initial placement of GE into ECM.

FIG. 5. In vitro rejuvenation of dedifferentiated muscle progenitor cells by anti-aging environment (AE1) condition. Primary cultures of myogenic progenitor cells were cultured overnight in dedifferentiation media to promote an exit from cell cycle and return to quiescence. Afterwards cells were cultured in control or anti-aging environment (+AE) in the presence of Opti-MEM containing 5% of either young (YM) or old mouse sera (OM) for 60 hours. BrdU was added 2 h prior to fixation for labeling dividing cells. Immunofluorescence was performed after fixation with the indicated antibodies: α-BrdU (red), α-eMHC (green) and Hoechst (blue) was used to label all nuclei. The myogenic potential of these cells was measured as their ability to proliferate (BrdU⁺) and to form de-novo eMHC⁺ myotubes. Control picture OM shows that old serum inhibited myogenic potential of these young progenitor cells cultured with control adhesion substrate. However, local release of Delta and Shh in combination with anti-TGF-β neutralizing antibody from the modified adhesion substrate (OM+AF) overrides the inhibition of myogenic potential imposed by the old serum and enhances regenerative capacity of these cells in both young, and importantly, old systemic milieu.

FIG. 6. Schematic of the experimental technique developed for dividing ECM adhesion substrates into different biologically-active geometric areas. Class chamber slides were pre-coated with 40 μg/mL ECM gel one day prior to experiments. To create separate environments, cloning cylinders were placed into the middle of each chamber during pre-coating. Pre-coated slides with cylinders were allowed to congeal overnight at room temperature, so that a seal between the interior and the exterior of the cloning cylinders was formed during the gelation of the ECM. 24 h later, growth factors (GF): basic-FGF (0.05 μg/ml), Follistatin (0.5 μg/ml) and α-TGF-β (1 μg/ml); and differentiation factors (DF): GDF-8/myostatin (0.1 μg/ml) IGF-1 (0.5 μg/ml) and TGF-β (0.02 μg/ml) were prepared separately in ECM/PBS solution (12 μg/ml) and placed inside different cylinders of each chamber. In order to maintain the seal formed, which is vital in preventing exchange of GF or DF between the interior and the exterior environments of the cylinders. Pressure equilibrium between the outside and inside environments of the cylinders was maintained while adding factors. This preparation was kept overnight (˜24 hours) at 4° C. to allow integration of factors into the ECM layer. Afterwards, all residual liquid inside chambers and cylinders was aspirated and cloning cylinders were removed from the chambers, leaving behind areas of modified ECM with embedded GF and DF. Myoblasts were re-suspended into desired media (GM, DM or NM), seeded under each experimental condition into corresponding chambers and cultured for 48 hours. Uniform cell adhesion is allowed with this method, because no damage of the ECM is caused when cylinders are removed prior to cellular seeding. In order to measure cell proliferation or differentiation by immunofluorescence, BrdU was added to cultured media 2 hours prior to cell fixation in order to label replicating cells. Cells were fixed with 70% EtOH in PBS alter 48 hours of specific culture conditions.

### DETAILED DESCRIPTION OF THE PREFERRED EMBODIMENTS

I. Definitions and Abbreviations

The abbreviations used herein generally have their conventional meaning within the chemical and biological arts.

“Composition of the invention.” as used herein refers to the compositions discussed herein, pharmaceutically acceptable salts and prodrugs of these compositions.

Where substituent groups are specified by their conventional chemical formulae, written from left to right, they equally encompass the chemically identical substituents, which would result from writing the structure from right to left, e.g. —CH₂O— is intended to also recite —OCH₂—.

By “effective” amount of a drug, formulation, or permeant is meant a sufficient amount of a active agent to provide the desired local or systemic effect. A “Topically effective,” “Cosmetically effective,” “pharmaceutically effective,” or “therapeutically effective” amount refers to the amount of drug needed to effect the desired therapeutic result.

The term “pharmaceutically acceptable salts” is meant to include salts of the compounds of the invention which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfoinic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science 66: 1-19 (1977)). Certain specific compounds of the present invention contain both basic and acidic functionalities that allow the compounds to be converted into either base or acid addition salts.

The neutral forms of the compounds are preferably regenerated by contacting the salt with a base or acid and isolating the parent compounds in the conventional manner. The parent form of the compound differs from the various salt forms in certain physical properties, such as solubility in polar solvents.

In addition to salt forms, the present invention provides compounds which are in a prodrug form. Prodrugs of the compounds or complexes described herein readily undergo chemical changes under physiological conditions to provide the compounds of the present invention. Additionally, prodrugs can be convened to the compounds of the present invention by chemical or biochemical methods in an ex vivo environment.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

The term “pharmaceutically acceptable carrier” or “pharmaceutically acceptable vehicle” refers to any formulation or carrier medium that provides the appropriate delivery of an effective amount of a active agent as defined herein, does not interfere with the effectiveness of the biological activity of the active agent, and that is sufficiently non-toxic to the host or patient. Representative carriers include water, oils, both vegetable and mineral, cream bases, lotion bases, ointment bases and the like. These bases include suspending agents, thickeners, penetration enhancers, and the like. Their formulation is well known to those in the art of cosmetics and topical pharmaceuticals. Additional information concerning carriers can be found in Remington: The Science and Practic of Pharmacy, 21st Ed., Lippincott, Williams & Wilkins (2005) which is incorporated herein by reference.

“Pharmaceutically acceptable topical carrier” and equivalent terms refer to pharmaceutically acceptable carriers, as described herein above, suitable for topical application. An inactive liquid or cream vehicle capable of suspending or dissolving the active agent(s), and having the properties of being nontoxic and non-inflammation when applied to the skin, nail, hair, claw or hoof is an example of a pharmaceutically-acceptable topical carrier. This term is specifically intended to encompass carrier materials approved for use in topical cosmetics as well.

The term “pharmaceutically acceptable additive” refers to preservatives, antioxidant, fragrances, emulsifiers, dyes and excipients known or used in the field of drug formulation and that do not unduly interfere with the effectiveness of the biological activity of the active agent, and that is sufficiently non-toxic to the host or patient. Additives for topical formulations are well-known in the art, and may be added to the topical composition, as long as they are pharmaceutically acceptable and not deleterious to the epithelial cells or their function. Further, they should not cause deterioration in the stability of the composition. For example, inert fillers, anti-irritants, tackifiers, excipients, fragrances, opacifiers, antioxidants, gelling agents, stabilizers, surfactant, emollients, coloring agents, preservatives, buffering agents, other permeation enhancers, and other conventional components of topical or transdermal delivery formulations as are known in the art.

The term “excipients” is conventionally known to mean carriers, diluents and/or vehicles used in formulating drug compositions effective for the desired use.

The term “autologous cells”, as used herein, refers to cells which are person's own genetically identical cells.

The term “heterologous cells”, as used herein, refers to cells which are not person's own and are genetically different cells.

The term “stem cells”, as used herein, refers to cells capable of differentiation into other cell types, including those having a particular, specialized function (i.e., terminally differentiated cells, such as erythrocytes, macrophages, etc.). Stem cells can be defined according to their source (adult/somatic stem cells, embryonic stem cells), or according to their potency (totipotent, pluripotent, multipotent and unipotent).

The term “unipotent”, as used herein, refers to cells can produce only one cell type, but have the property of self-renewal which distinguishes them from non-stem cells.

The term, “multipotent”, or “progenitor”, as used herein, refers to cells which can give rise to any one of several different terminally differentiated cell types. These different cell types are usually closely related (e.g. blood cells such as red blood cells, white blood cells and platelets). For example, mesenchymal stem cells (also known as marrow stromal cells) are multipotent cells, and are capable of forming osteoblasts, chondrocytes, myocytes, adipocytes, neuronal cells, and β-pancreatic islets cells.

The term “pluripotent”, as used herein, refers to cells that give rise to some or many, but not all, of the cell types of an organism. Pluripotent stem cells are able to differentiate into any cell type in the body of a mature organism, although without reprogramming they are unable to de-differentiate into the cells from which they were derived. As will be appreciated, “multipotent”/progenitor cells (e.g., neural stem cells) have a more narrow differentiation potential than do pluripotent stem cells. Another class of cells even more primitive (i.e., uncommitted to a particular differentiation fate) than pluripotent stem cells are the so-called “totipotent” stem cells.

The term “totipotent”, as used herein, refers to fertilized oocytes, as well as cells produced by the first few divisions of the fertilized egg cell (e.g., embryos at the two and four cell stages of development). Totipotent cells have the ability to differentiate into any type of cell of the particular species. For example, a single totipotent stem cell could give rise to a complete animal, as well as to any of the myriad of cell types found in the particular species (e.g., humans). In this specification, pluripotent and totipotent cells, as well as cells with the potential for differentiation into a complete organ or tissue, are referred as “primordial” stem cells.

The term “dedifferentiation”, as used herein, refers to the return of a cell to a less specialized state. After dedifferentiation, such a cell will have the capacity to differentiate into more or different cell types than was possible prior to re-programming. The process of reverse differentiation (i.e., de-differentiation) is likely more complicated than differentiation and requires “reprogramming” the cell to become more primitive. An example of dedifferentiation is the conversion of a myogenic progenitor cell, such as early primary myoblast, to a muscle stem cell or satellite cell.

The term “anti-aging environment”, as used herein, is an environment which will cause a cell to dedifferentiate, or to maintain its current state of differentiation. For example, in an anti-aging environment, a myogenic progenitor cell would either maintain its current state of differentiation, or it would dedifferentiate into a satellite cell.

A “normal” stem cell refers to a stem cell (or its progeny) that does not exhibit an aberrant phenotype or have an aberrant genotype, and thus can give rise to the full range of cells that be derived from such a stein cell. In the context of a totipotent stem cell, for example, the cell could give rise to, for example, an entire, normal animal that is healthy. In contrast, an “abnormal” stem cell refers to a stem cell that is not normal, due, for example, to one or more mutations or genetic modifications or pathogens. Thus, abnormal stem cells differ from normal stem cells.

A “growth environment” is an environment in which stem cells will proliferate in vitro. Features of the environment include the medium in which the cells are cultured, and a supporting structure (such as a substrate on a solid surface) if present.

“Growth factor” refers to a substance that is effective to promote the growth of stem cells and which, unless added to the culture medium as a supplement, is not otherwise a component of the basal medium. Put another way, a growth factor is a molecule that is not secreted by cells being cultured (including any feeder cells, if present) or, if secreted by cells in the culture medium, is not secreted in an amount sufficient to achieve the result obtained by adding the growth factor exogenously. Growth factors include, but are not limited to, basic fibroblast growth factor (bFGF), acidic fibroblast growth factor (aFGF), epidermal growth factor (FGF), insulin-like growth factor-I (IGF-I), insulin-like growth factor-II (IGF-II), platelet-derived growth factor-AB (PDGF), and vascular endothelial cell growth factor (VEGF), activin-A, and bone morphogenic proteins (BMPs), insulin cytokines, chemokines, morphogents, neutralizing antibodies, other proteins, and small molecules.

The term “differentiation factor”, as used herein, refers to a molecule that induces a stem cell to commit to a particular specialized cell type.

“Extracellular matrix” or “matrix” refers to one or more substances that provide substantially the same conditions for supporting cell growth as provided by an extracellular matrix synthesized by feeder cells. The matrix may be provided on a substrate. Alternatively, the component(s) comprising the matrix may be provided in solution. Components of an extracellular matrix can include laminin, collagen and fibronectin.

The term “regenerative capacity”, as used herein refers to conversion of stem cell into dividing progenitor cell and differentiated tissue-specific cell.

The term “rejuvenation”, as used herein, refers to changing the regenerative responses of a stem cell such that the stem cell successfully or productively regenerates tissues in organs even if such organs and tissues are old and the stem cells are old.

II. Introduction

The present invention provides an ex vivo composition which includes an anti-aging, growth or differentiation environment for a cell. These compositions include an extracellular matrix and at least one anti-aging factor, growth factor, or differentiation factor. Methods of making the compositions, and methods of using the compositions for providing stem cells to a patient, or treating a condition in a patient, are also encompassed by this invention.

III. The Compositions

The invention provides methods for engineering artificial adhesion substrate material, which contains molecularly defined ECM which contain natural protein components, such as laminin, collagen, fibronectin. In an exemplary embodiment, the ECM can contain synthetic components. In an exemplary embodiment, the natural components of the ECM will provide attachment and signaling to stem cells, and the chemical components will provide optimal gelification temperatures as well as rigidity, strength and other mechanical properties for ex vivo and in vivo applications.

In a first aspect, the invention provides a modified adhesion substrate composition including an extracellular matrix and at least one anti-aging factor, wherein the composition provides an anti-aging environment for the conversion of at least one progenitor cell to a stem cell. In an exemplary embodiment, the composition also includes a stem cell. In another exemplary embodiment, the stem cell is a satellite cell. In another exemplary embodiment, the anti-aging factor is a member selected from Table 1.

TABLE 1
Factors used in the modified adhesion substrate compositions

| | | Growth- | Differentiation- | | |
|---|---|---|---|---|---|
| | | promoting | promoting | | |
| | Effects on | Factor | Factor | Anti-Aging | |
| Factor | myogenesis | combinations | combinations | Combinations | References |
| FGF-2 | Enhanced | + | | + | (Maley et al., Exp. Cell Res., |
| | proliferation | | | | 211(1): 99-107(1994)), (Miller et |
| | of myogenic | | | | al., Am. J. Physiol Cell Physiol, |
| | progenitor | | | | 278(1): C174-C181 (2000))) |
| | ceils | | | | |
| GDF-8/ | Inhibition of | | + | | (McCroskery et al., J. Cell |
| Myostatin | proliferation | | | | Biol., 162, (6): 1135-1147 (2003)), |
| | of myogenic | | | | (McPherron et al., Nature, |
| | progenitor | | | | 387(6628): 83-90 (1997)), |
| | cells | | | | (Thomas et al., J. Biol. Chem., |
| | | | | | 275(51): 40235-40243 (2000)), |
| | | | | | (Zimmers et al., Science, |
| | | | | | 296(5572): 1486-1488 (2002)) |
| Follistatin | Inhibition of | + | | + | (Armand et al., Dev. Dyn., |
| | Myostatin | | | | 227(2): 256-265 (2003)) |
| IGF-1 | Enhanced | | + | | (Bodine et al., Nat. Cell Biol., |
| | differentiation | | | | 3(11): 1014-1019 (2001)), |
| | and increased | | | | (Downward, Semin. Cell |
| | myofiber | | | | Dev. Biol.. 15(2): 177-182 |
| | size/mass | | | | (2004)), (Florini et al., |
| | | | | | Endocr. Rev., 17(5): 481-517 |
| | | | | | (1996)), (Heszele et al., |
| | | | | | Endocrinology, 145(11): 4803- |
| | | | | | 4805 (2004)), (Lawlor et al.,, |
| | | | | | J. Cell Biol., 151(6): 1131-1140 |
| | | | | | (2000)), (Sandri et al., Cell. |
| | | | | | 117(3): 399-412 (2004)), |
| | | | | | (Shavlakadze et al., |
| | | | | | Neuromuscul. Disord., |
| | | | | | 15(2): 139-146(2005)) |
| TGF-β | Generic | | + | | (Derynck et al., Nature, |
| | inhibitor of | | | | 425(6958): 577-584 (2003)), |
| | cell cycle | | | | (Jakubowiak et al., |
| | progression, | | | | J. Biol. Chem., 275, (51): 40282- |
| | promotes | | | | 40287 (2000)), (Massague et |
| | differentiation | | | | al., Cell, 103(2): 295-309 |
| | and wound | | | | (2000)), (Massague et al.. |
| | healing | | | | Genes Dev., 14(6): 627-644 |
| α-TGF-β | Neutralization | + | | + | (2000)) |
| | of TGF-β | | | | |
| | activity | | | | |
| DLL4 | Notch ligand, | | | + | (Conboy et al., Science, |
| | enhances | | | | 302(5650): 1575-1577 (2003)), |
| | activation of | | | | (Conboy et al., Dev. Cell, 3 |
| | Notch | | | | (3): 397-409 (2002)) |
| | | | | | (Conboy et al., Cell Cycle, |
| | | | | | 4(3): 407-410 (2005)), Wagers |
| | | | | | et al., Cell, 122(5): 659-667 |
| | | | | | (2005)) |
| Shh | Enhanced | | | + | (Conboy et al., Science, |
| | muscle repair | | | | 302(5650): 1575-1577 (2003)), |
| | in vivo | | | | Conboy et al., Cell Cycle, |
| | | | | | 4(3): 407-410 (2005)), Wagers |
| | | | | | et al., Cell, 122(5): 659-667 |
| | | | | | (2005)) |

In another exemplary embodiment, the anti-aging factor is a member selected from DLL4, Shh, α-TGF-β, b-FGF and follistatin. In another exemplary embodiment the composition comprises at least three anti-aging factors. In another exemplary embodiment, the anti-aging factors are DLL4, Shh and α-TGF-β. In another exemplary embodiment, DLL4 is present in the composition in a concentration of from about 0.1 μg/mL to about 1 μg/mL, Shh is present in the composition in a concentration of from about 0.1 μg/ml to about 1 μg/mL and α-TGF-β is present in the composition in a concentration of from about 1 μg/mL to about 50 μg/mL. In another exemplary embodiment, DLL4 is present in the composition in a concentration of about 0.5 μg/mL, Shh is present in the composition in a concentration of about 0.5 μg/ml and α-TGF-β is present in the composition in a concentration of about 10 μg/mL.

In an exemplary embodiment, the composition comprises at least five anti-aging factors. In another exemplary embodiment, the anti-aging factors are DLL4, Shh, α-TGF-β, b-FGF and follistatin. In an exemplary embodiment, DLL4 is present in the composition in a concentration of from about 0. 1 μg/mL to about 1 μg/mL, Shh is present in the composition in a concentration of from about 0.1 μg/mL, to about 1 μg/mL, α-TGF-β is present in the composition in a concentration of from about 1 μg/mL to about 50 μg/mL, b-FGF is present in the composition in a concentration of from about 0.01 μg/mL to about 0.1 μg/mL and follistatin is present in the composition in a concentration of from about 1 μg/mL to about 10 μg/mL in an exemplary embodiment, DLL4 is present in the composition in a concentration of about 0.5 μg/mL, Shh is present in the composition in a concentration of about 0.5 μg/mL, α-TGF-β is present in the composition in a concentration of about 10 μg/mL, b-FGF is present in the composition in a concentration of about 0.05 μg/mL and follistatin is present in the composition in a concentration of about 0.5 μg/mL.

In a second aspect, the invention provides a modified adhesion substrate composition including an extracellular matrix and at least one growth factor, wherein the composition provides a growth environment for a cell. In an exemplary embodiment, the composition also includes a stem cell or a progenitor cell. In another exemplary embodiment, the stem cell is a satellite cell. In another exemplary embodiment, the progenitor cell is a myoblast. In another exemplary embodiment, the growth factor is a member selected from Table 1. In another exemplary embodiment, the growth factor is a member selected from basic-FGF, follistatin and α-TGF-β. In another exemplary embodiment the composition comprises at least three anti-aging factors. In another exemplary embodiment, basic-FGF is present in the composition in a concentration of from about 0.01 μg/mL to about 0.1 μg/mL, follistatin is present in the composition in a concentration of from about 0.1 μg/mL to about 1 μg/mL and α-TGF-β is present in the composition in a concentration of from about 1 μg/mL to about 50 μg/mL. In another exemplary embodiment, basic-FGF is present in the composition in a concentration of about 0.5 μg/mL, follistatin is present in the composition in a concentration of about 0.5 μg/mL and β-TGF-β is present in the composition in a concentration of about 10 μg/mL. In another exemplary embodiment, the composition also includes differentiation media.

In a third aspect, the invention provides a modified adhesion substrate composition including an extracellular matrix and at least one differentiation factor, wherein the composition provides a differentiation environment for a cell. In an exemplary embodiment, the composition also includes a stem cell or a progenitor cell. In another exemplary embodiment, the stem cell is a satellite cell. In another exemplary embodiment, the progenitor cell is a myoblast. In another exemplary embodiment, the growth factor is a member selected from Table 1. In another exemplar embodiment, the differentiation factor is a member selected from myostatin, IGF-1 and TGF-β. In another exemplary embodiment, the composition includes at least three anti-aging factors. In another exemplary embodiment, myostatin is present in the composition in a concentration of from about 0.01 μg/mL to about 1 μg/mL, IGF-1 is present in the composition in a concentration of from about 0.1 μg/mL to about 1 μg/mL and TGF-β is present in the composition in a concentration of from about 0.01 μg/mL to about 0.1 μg/mL. In another exemplary embodiment, myostatin is present in the composition in a concentration of about 0.1 μg/mL, IGF-1 is present in the composition in a concentration of about 0.5 μl/mL and TGF-β is present in the composition in a concentration of about 0.02 μg/mL. In another exemplary embodiment, the composition also includes growth media.

III. The Methods

According to another aspect, the invention provides a method of treating a disease or condition comprising administering to a patient in need of treatment a therapeutically effective amount of a modified adhesion substrate composition of the invention. In an exemplary embodiment, the disease or condition is a skeletal muscle disorder.

According to yet another aspect, the invention provides a method of treating an injury in a patient, comprising administering a modified adhesion substrate composition of the invention to the patient. In an exemplary embodiment, the injury is a muscle injury.

According to another aspect, the invention provides a method of rejuvenating stem cells in a patient, comprising contacting a stem cell with a modified adhesion substrate composition of the invention, thereby rejuvenating the stem cell.

According to still another aspect, the invention provides a method of transplanting stem cells into a patient, comprising administering a stem cell with a modified adhesion substrate composition of the invention, thereby transplanting the stem cell into the patient.

According to still another aspect, the invention provides a method of transplanting stem cells into a patient, comprising administering autologous or heterologous stem cell with a modified adhesion substrate composition of the invention to the patient, thereby enhancing the performance of the transplanted autologous or heterologous stem cell in the patient.

According to still another aspect autologous or heterologous stem cells will be encapsulated in a modified adhesion substrate composition of the invention that include two layers of sequentially activated environments. These sequentially activated environments are members selected from a growth environment, a differentiation environment and an anti-aging environment. This invention can be used for deliberate induction first of cell expansion followed by tissue-specific differentiation. The anti-aging modifications of the modified adhesion substrate composition of the invention will be used in older patients.

Various aspects of the present invention will be further illustrated by the following non-limiting examples.

### EXAMPLES

General

C57B1/6 mice were obtained from Jackson Laboratories and housed at UC Berkeley Animal Care Facility. Antibodies to BrdU, to eMHC and nuclear stain Hoechst were obtained from Abeam Inc. (Cambridge, Mass.), Vector Laboratories (Burlingame, Calif.) and Sigma (St. Louis, Mo.), respectively. Secondary antibodies were obtained from Molecular Probes (Eugene, Oreg.). TGF-β, GCDF-8, FGF-2, α-TGF-β and IGF-1 were all obtained from R&D Labs (Minneapolis, Minn.). Follistatin was obtained from Sigma (St. Louis, Mo.). Ham's F10 and DMEM media and penicillin/streptomycin were obtained from Mediatech Inc. (Herndon, Va.) and OptiMEM media and Fetal Bovine Serum (FBS) were from Invitrogen Corp. (Carlsbad, Calif.). Horse Serum (HS) was also obtained from Mediatech Inc. (Herndon, Va.). Phosphate Buffer Solution (PBS) was obtained from Fisher Scientific (Fairlawn, N.J.) and ECM gel from Engelbreth Holm-Swarm (EHS) mouse sarcoma from Sigma (St. Louis, Mo.). This ECM gel contains collagens, non-collagenous glycoproteins and proteoglycans. Precisely, its major component is laminin, and it also contains collagen type IV, heparan sulfate proteoglycan, entactin and other minor components. Two and four chamber culture slides were obtained from BD Biosciences (Bedford, Mass.) and cloning cylinders were obtained from VWR International.

### Example 1

Preparation of Growth Environment, Differentiation Environment Compositions

Progenitor Cell Isolation from Injured Muscle

Both muscle injury and acquisition of muscle progenitor cells from myofiber fragments were performed as previously published (Conboy & Rando 2002). Briefly, 3 days after muscle injury, hind leg muscle was dissociated into myofibers, which were cultured overnight, during which time activated satellite cells give rise to colonies of myogenic progenitor cells. These cells called myoblasts were then expanded and used in this work. Myofibers as well as myoblasts were cultured on ECM-coated plates (8 μg/mL)) in growth medium (GM), differentiation medium (DM), or neutral medium (NM).

Media Preparation

Growth media (GM) consisted of Ham's F10+20% FBS+FGF-2 (5 ng/ml)+1% penicillin/streptomycin, differentiation media (DM) consisted of DMEM+2% HS+1% penicillin/streptomycin and neutral media (NM) consisted of OptiMEM+5% FBS+1% penicillin/streptomycin. Dedifferentiation media consisted of OptiMEM+1% FBS+1% penicillin/streptomycin.

Cell Placement

Prior to seeding cells into each chamber, all residual liquid inside chambers and cylinders was aspirated and afterwards cloning cylinders were removed from the chambers, leaving behind areas of modified ECM with embedded GF and DF. Myoblasts were re-suspended into desired media (GM, DM or NM), seeded under each experimental condition into corresponding chambers and cultured for 48 hours. For examining anti-aging effects, progenitor cells were first dedifferentiated into stem cells by culturing them in de-differentiation media for 24 hours. Then cells were plated into anti-aging environment modified adhesion substrates and cultured for 60 hours which allows their rejuvenation in the presence of old mouse serum.

Slide Preparation

Two-chamber slides were pre-coated with ECM one day prior to experiments. To create a separate environment, cloning cylinders were placed into the middle of each chamber during pre-coating. The pre-coated slide with cylinder was allowed to congeal overnight at room temperature. A seal between the interior and the exterior of the cloning cylinders was formed during the gelation of the ECM as the cylinders penetrated the liquid ECM due to gravity. This seal is vital in preventing exchange of GF or DF between the interior and the exterior environments of the cylinders. To facilitate uniform cell adhesion and proliferation, the seal must also be such that it does not damage the ECM when cylinders are removed prior to cellular seeding. To achieve these parameters, different concentrations of ECM were tested and a concentration of 40 μg/mL was finally selected.

In order to maintain the seal formed, pressure equilibrium between the outside and inside environments of the cylinders was maintained while adding factors. This allows us to confine the factors to a specific area on the slide determined by the geometry and size of the cloning cylinders. Different cloning cylinders were also tested and those with the best surface finishing of the cross section (Scienceware cloning cylinders) produced the best seal and thus the best geometric boundary.

For control experiments, four-chamber slides were used and pre-coated in similar fashion without the cylinders.

Immunofluorescence Analysis

In order to measure cell proliferation or differentiation by immunofluorescence as previously described (Conboy et al., Dev. Cell, 3 (3):397-409 (2002)), myoblasts were fixed with 70% EtOH in PBS alter 36 or 48 hours of specific culture conditions. BrdU was added to cultured media 2 hours prior to cell fixation in order to label replicating cells. After fixing cells, they were washed with staining buffer (PBS+1% FBS+0.5% Na azide) and permeabilized in staining buffer containing 0.25% Triton X-100. Afterwards cells were incubated with antibodies specific for both proliferation (BrdU) and differentiation (eMHC) for one hour at room temperature. Hoechst stain was added during secondary antibody incubation. For BrdU detection, cells were incubated with 4M HCl at room temperature prior to permeabilization, to denature DNA. α-eMHC was used at 1:25 hybridoma supernatant dilution and α-BrdU at 2.5 μg/mL. Secondary antibodies and Hoechst were used at 1:500 hybridoma supernatant dilution.

Quantification and Statistics

Cells were counted from triplicate experiments with at least 300 cells per experiment. Cells expressing BrdU proliferation marker were counted as proliferating cells. Cells expressing eMHC and containing 2 or less nuclei per fiber were counted as early-differentiating cells D1 and those with more than 2 nuclei per fiber were D2, Statistical significance confidence intervals were analyzed with p-value test (Anova: Single Factor) and error bars.

Growth/Proliferation Environment (GE) Conditions

A Growth/Proliferation Environment (GE) was created through adding, the following molecules to the ECM in the following concentrations: basic-FGF (0.05 μg/mL), Follistatin (0.5 μg/mL) and α-TGF-β (10 μL/mL). These factors were each added to ECM/PBS solution (12 μg/ml) and then the mixed solution was added to the ECM described earlier. This preparation was kept overnight (˜24 hours) at 4° C. to allow integration of factors into the ECM layer.

Differentiation Environment (DE) Conditions

A Differentiation Environment (DE) was created through adding the following molecules to the ECM in the following concentrations: GDF-8/myostatin (0.1 μg/mL), IGF-1 (0.5 μg/mL) and TGF-β (0.02 μg/mL). These factors were each added to ECM/PBS solution (12 μg/mL) and then the mixed solution was added to the ECM described earlier. This preparation was kept overnight (˜24 hours) at 4° C. to allow integration of factors into the ECM layer.

### Example 2

Testing of Growth Environment, Differentiation Environment Compositions

Different combinations of GE and DE (Table 1) were embedded into ECM gel, to examine whether these factors would be able to override the cell fate imposed by the aforementioned media, and at the same time, whether a clearly defined boundary between cells with alternative myogenic cell fates could be created by their adhesion to modified ECM substrates which contain either GF or DF.

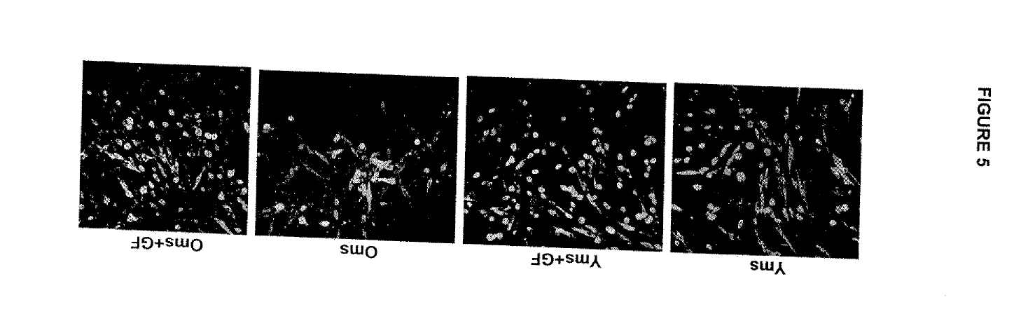

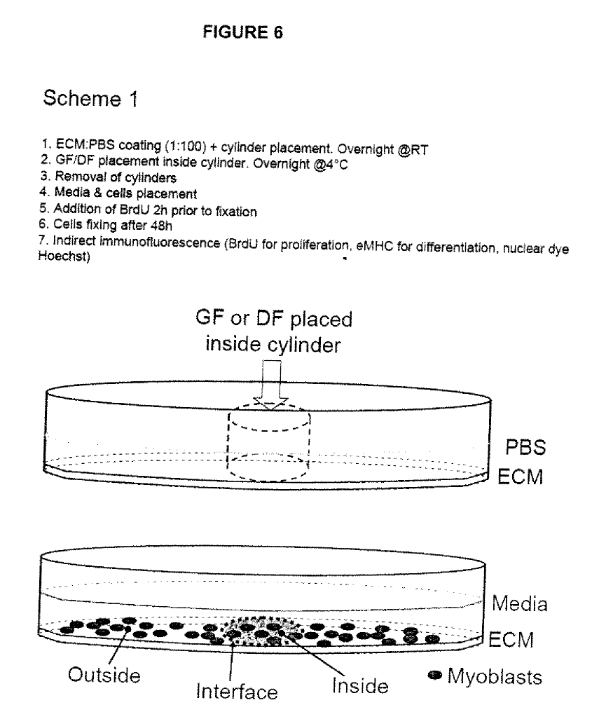

To achieve these goals, the experimental technique described in Example 1 and summarized in FIG. 6 was created. Briefly, ECM gels were divided into different geometric areas using cloning cylinders and mixtures of either GF or DF were placed inside them, creating modified areas of ECM which contained factors, versus the non-modified ECM areas. Afterwards cells were uniformly seeded onto the whole ECM substrate, so that after approximately one hour cells adhered to both non-modified and modified ECM areas and shared the same media. Adherence of cells to non-modified and modified ECM was simultaneous and there was no difference in cell survival. Experiments with uniformly embedded GF or DF into the whole ECM area have also been carried out as positive controls.

Manipulating Cell Fate in Growth Media (GM)

First, we have examined the behavior of cells in GM without any factors embedded in ECM. Consistent with previously published results (Conboy et al. Cell Cycle 4(3):407-410 (2005)), (Morgan et al., Int J. Biochem. Cell Biol. 35(8):1151-1156 (2003)), cells rapidly proliferate and do not differentiate in GM, i.e. they incorporate BrdU and only less than 1% express the marker of differentiated myotubes, eMHC (FIG. 1A-GM, quantified in FIG. 1B-GM). In contrast, embedded DF in ECM successfully promote myogenic differentiation of primary myoblasts (FIG. 1A-GM+DF), as shown by their reduced proliferation and enhanced expression of eMHC. Interestingly, such directed differentiation occurs even in the presence of highly mitogenic GM, which contains 20% FBS and FGF-2 (FIG. 1A-GM+DF, quantified in FIG. 1B-GM+DF). FIG. 1B demonstrates quantification of multiple experiments and statistically shows that the effects caused by DF-modified ECM significantly promotes differentiation of myogenic cells exposed to mitogenic media. Specifically, cells attached to areas of DF-modified ECM show higher expression of eMHC⁺ (number of cells at early stage of differentiation, D1, significantly rises from 0.5% to 11.8%: p value=0.003). In addition, even though cells exposed to DF-modified ECM continue to proliferate, the rate was slower (FIG. 1B: Number of proliferating cells P drops from 20.9% in GM to 9.6% in GM+DF; p value=0.077) and a higher percentage of these cells expresses the differentiation marker, eMHC (FIG. 1B-GM+DF: D1=11.8%). Thus, these findings reveal that it is possible to force the differentiation of cells under proliferative media conditions through DF-modified ECM substrates.

Moreover, geometric control of cell fate determination was achieved, as clearly shown in FIG. 1C, where an obvious interface between eMHC⁺ and eMHC⁻ myogenic progenitor cells cultured under identical media conditions was created by exposing these cells to the different regions of ECM substrate (with versus without DF).

Manipulating Cell Fate in Differentiation Media (DM)

To confirm and extrapolate these findings, testing was conducted to determine whether the reciprocal cell fate determination could also be achieved within this experimental system. Specifically, cells were cultured in DM with non-modified versus GF-modified ECM. Unsurprisingly, fusion competent myoblasts terminally differentiate and form eMHC⁺ myotubes when cultured in DM (Conboy et al., Dev. Cell, 3 (3):397-409 (2002)), (Conboy et al., Cell Cycle, 4(3):407-410 (2005)), (Morgan et al., Int. J. Biochem. Cell Biol., 35(8):1151-1156 (2003)) (FIG. 2A-DM, quantified in FIG. 2B-DM). In contrast, FIG. 2A-DM+GF shows that much fewer eMHC⁺ myotubes are formed in the area where cells are exposed to GF, as compared to the area devoid of GF. Additionally, a significant fraction of cells incorporate BrdU (FIG. 2A-DM+GF), thus overcoming the effect imposed by highly differentiating media when attached to GF-modified ECM substrates.

Both the robustness and reproducibility of the aforementioned regulation of cell fate by GF-signaling from specific areas of ECM were confirmed by the quantification of at least three replicated experiments, as illustrated in FIG. 2B-DM+DF. Proliferation (P) dramatically increases from 0.6% in DM to 16.7% in DM+GF (p value=0.003) and the number of cells at early stage of differentiation significantly drops from 18.9% to 2.9% (p value=0.004).

Notably, similar to the data shown in FIG. 1C, a clearly defined interface was created between the region of modified ECM, which contained embedded GF, and the non-modified control ECM area, thus allowing cells with different fates coexist in the same culture medium (FIG. 2C). This interface is discernable not only because some cells incorporate BrdU and some instead form myotubes, but also because of different cell densities. Specifically, there are approximately four times more cells in the area of ECM embedded with GF.

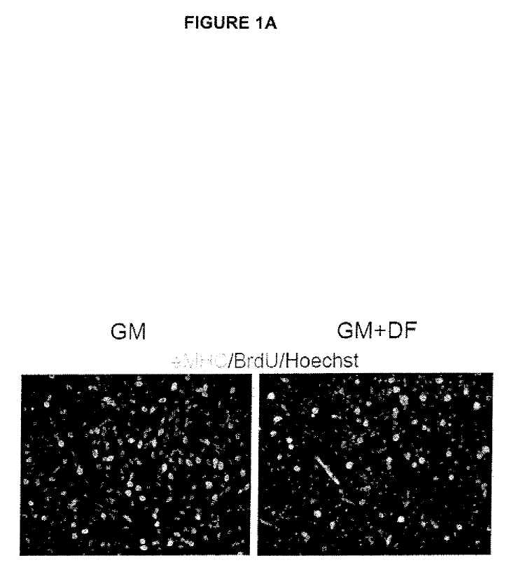

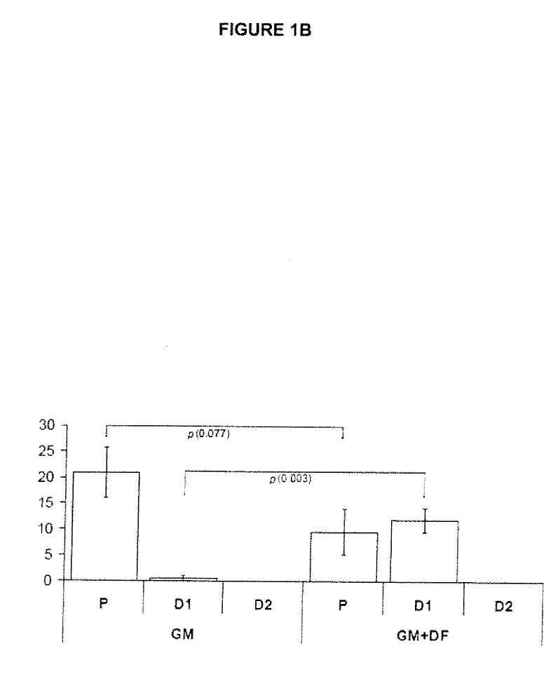

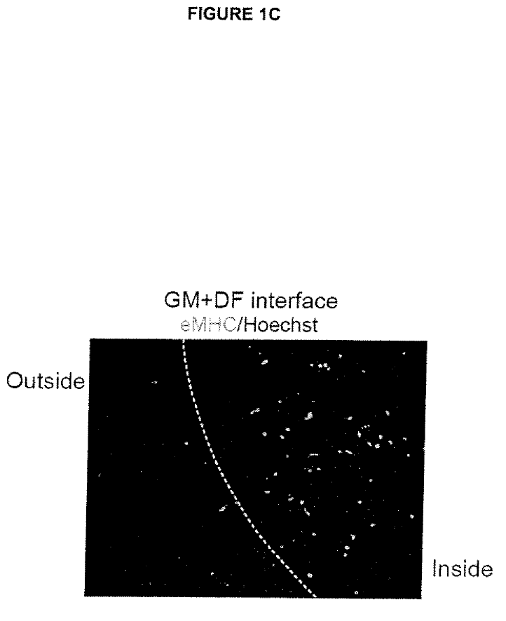

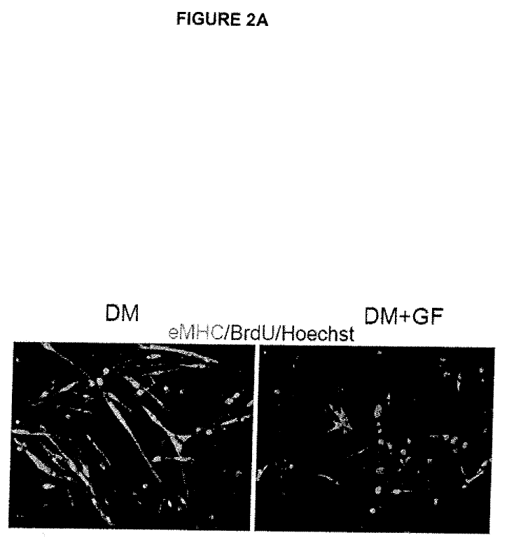

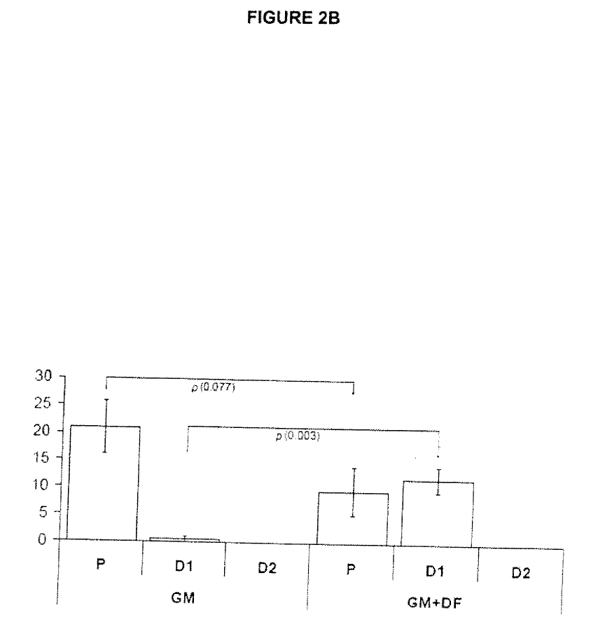

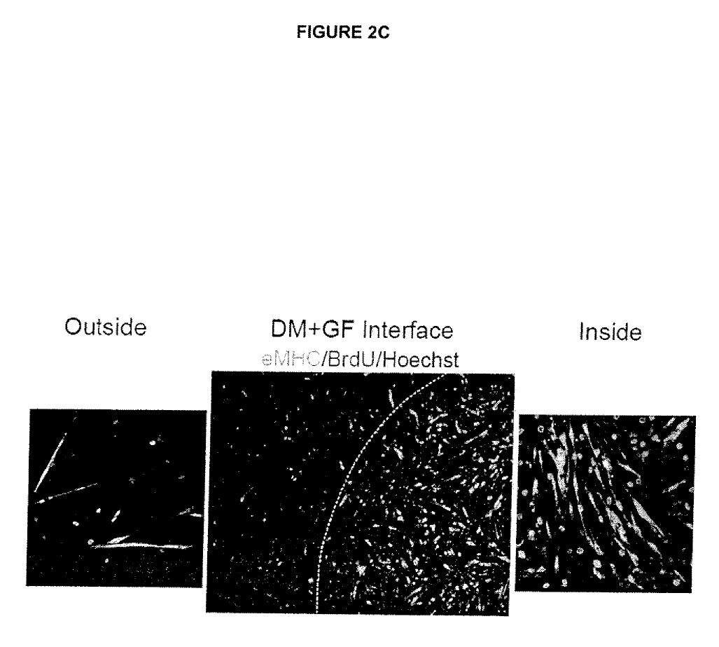

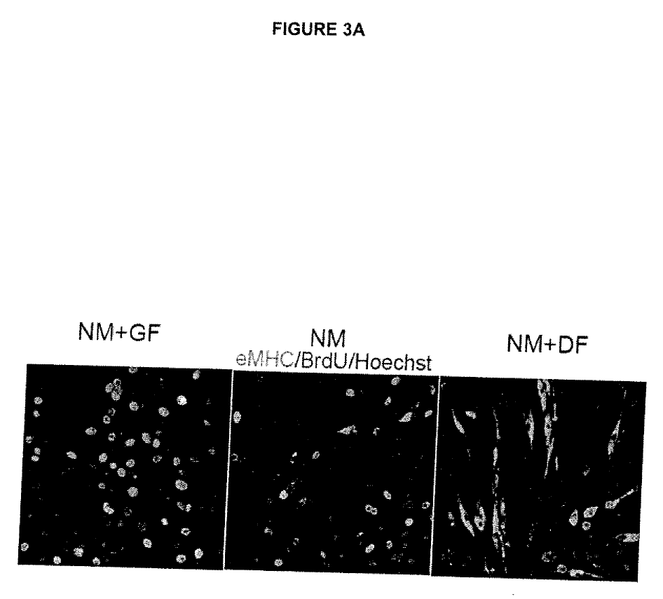

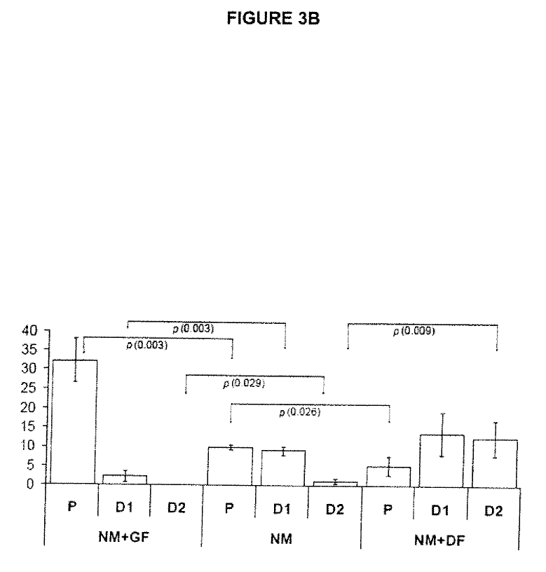

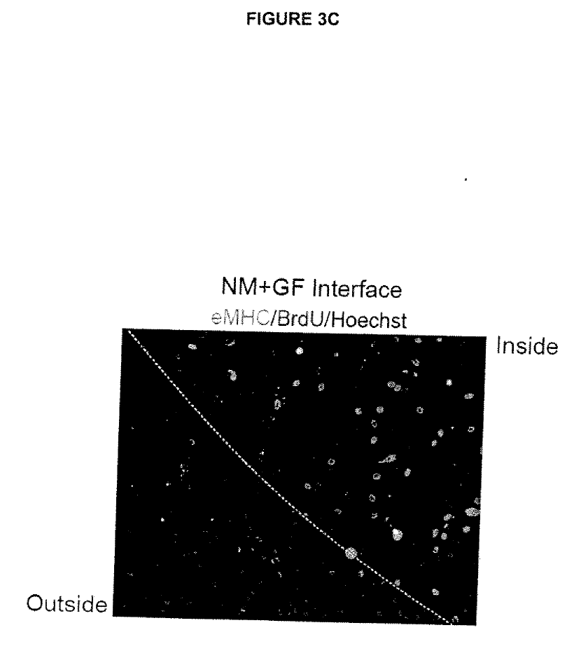

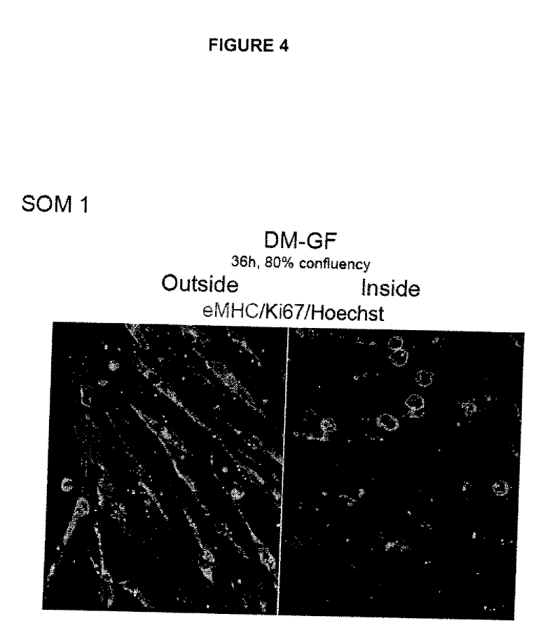

It is well known that plating myoblasts at high density will lead to exit of cell cycle and differentiation, even in the presence of GM (Conboy et al., Dev. Cell, 3 ( 3):397-409 (2002)), (Morgan et al., Int. J. Biochem. Cell Biol. 35(8):1151-1156(2003)). Thus, we decided to test whether we can inhibit differentiation under that specific condition. Even under high cell density (80% confluency), myogenic differentiation is delayed by GF-modified ECM, although not completely avoided (FIG. 4). Remarkably, when plated at high density, cells attached to GF-modified ECM area show higher levels of both proliferation and differentiation (FIG. 4). Therefore, as cell numbers increase differentiation seems inescapable, despite initial placement of GF into ECM substrate.

Manipulating Cell Fate in Neutral Media (NM)

After characterizing the effect of modified ECM compositions on cell fate in strongly differentiating or mitogen media, we tested our GF- and DF-modified ECM substrates in NM conditions. As expected, in NM condition myoblasts slowly proliferate and infrequently produce eMHC⁺ terminally differentiated cells, which usually have no more than one or two nuclei (FIG. 3A-NM and quantified in FIG. 3B-NM: fraction of proliferation cells P=9.8% and cells at earlv stage of differentiation D1=9.1%). This verifies that NM does not impose any strong determination of cell fate. Since proliferation and differentiation overlap during tissue repair, neutral media conditions might mimic the environment of regenerating muscle. As shown in FIG. 3A-NM+GF and quantified in FIG. 3B-NM+GF, cells cultured on modified ECM with embedded GF robustly proliferate (fraction of proliferating cells P=32.2%) and do not significantly differentiate (fraction of cells at early stage of differentiation D1=2.3%). This is confirmed by the robust incorporation of BrdU and absence of eMHC staining. In parallel myoblasts attached to modified ECM containing embedded DF efficiently differentiate and lack proliferation (FIG. 3A, NM+DF, quantified in FIG. 3B, NM+DF; fraction of proliferating cells P=5.2%; fraction of cells at early stage of differentiation D1=13.7%; fraction of cells at late stage of differentiation D2=12.4%). As above, quantification of at least three replicated experiments demonstrated high reproducibility of this geometric regulation of myogenic cell fate determination (FIG. 3B).

Consistent with data shown above, FIG. 3C demonstrates a clearly defined interface between cells with different rates of myogenic proliferation cultured in identical media (NM), which was created by the exposure of cells to the geometrically embedded GF into ECM.

In summary, data presented in this work demonstrate that GF and DF geometrically placed in adhesion substrates of myogenic progenitor cells compete against culture media for myogenic cell fate determination. As expected embedding GF and DF into ECM substrate yields uniform and opposite effects on the proliferation and differentiation of myogenic progenitor cells. Importantly the magnitude of the effects on either proliferation or differentiation is identical between uniformly-modified SCM (FIGS. 1A, 2A, 3A) and the spatially-modified areas of SCM (FIGS. 1C, 2C, 3C). This strongly suggests that geometrically embedded factors do not significantly diffuse throughout the ECM and that their initial concentrations are not diluted. Similar effects on proliferation and differentiation of myoblasts have been observed when these GF and DF (listed in Table 1) were added directly to culture media. Thus, the biological activity of these factors remains the same whether they are embedded in ECM or added to culture media. However, unlike GF- or DE-modified ECM substrates, directly applied factors are, of course, not capable of creating a geometric boundary between cells with different fates coexisting in the same culture dish. There is no doubt that these factors added to media signal via their specific receptors on cells (Husmann et al., Cytokine Growth Factor Rev., 7(3%) 249-258 (1996)), (Wagers et al. Cell 122(5):659-667 (2005)), thus identical regulation of cell fate from ECM embedded factors shown here strongly suggest that these factors also signal to cells attached to specific areas of modified ECM.

This work demonstrates that myogenic cells at different stages of proliferation and differentiation can deliberately be orchestrated to coexist adjacent to each other on the same plate with identical culture media by their attachment to modified ECM substrates. Thus, cells with different fates and at different stages of cell cycle can interact, and their direct interactions can be studied in the experimental system developed. During embryonic organogenesis, as well as in regenerating adult tissues rapidly proliferating, and terminally differentiated cells coexist and signal via both multiple cell-cell contacts and soluble molecules. Therefore, our developed technique can be used to identify the important molecular cross-talk regulating cell fate determination in embryonic development and in adult tissue repair.

This work also demonstrates that once cells expand, new myotubes are likely to be formed more efficiently and robustly in the presence of GF-modified ECM (FIG. 5). Such data is physiologically significant, since the loss of muscle strength and mass, know as muscle atrophy, often accompanies old age and muscle dystrophies. Thus, cell transplantation under conditions that are known to result in muscle hypertrophy could be especially beneficial for aged or pathologic organs.

### Example 3

Satellite Cell Isolation from Resting Muscle

The protocol used or the isolation of satellite cells from resting muscle is as follows:
Dissect resting muscle and put it into a 15 mL tube containing chilly Collagenase Media (CM; DMEM+1% penicillin streptomycin (s/p)+0.2% collagenase (Sigma)). Transport it cold to the lab.
Digest the muscle: Incubate it in CM 1-2 h @37 C shaking the tube slightly
Pour all the content of the tube into an uncoated PS Petri dish
Aspirate CM carefully.
2× wash cells with 5 ml PBS @RT and aspirate it fast.
Dissociate fibers in 7 mL Neutral Media rich in FBS (NMr: OptiMEM+10% FBS+1% s/p; FBS protects cells from the remaining collagenase in the media, which could kill them). Pipette up/down with 10 mL pipette.
Pipette up/down with 5 mL pipette
Pipette up/down with Pasteur pipette
Take all NMr (which contains suspended cells+debris) and put it into a 15 mL tube
Spin it down for 30 s @1000 rpm
Carefully transfer supernatant (which contains cells and less amount of debris) to a new 15 ml tube
50 mL syringe hack and forth 25× using 18 ga needle
Filter solution with cell strainer into a new 50 mL tube
Spin solution down for 5′ @1200 rpm
Re-suspend cells (pellet at the bottom of the tube) in Growth Media (GM: Ham's F10+20% FBS+1% p/s+FGF-2 (5 ng/ml) and plate them into a ECM-coated dish
Incubate dish for between 2 and 4 h (give enough time to satellite cells for attaching)
Aspirate GM and add new GM: Satellite cells are ready to be used.

An Anti-Aging Environment (AE) was created through adding the following molecules to the ECM in the following concentrations: DLL4 (0.5 μg/mL). Shh (0.05 μg/mL) and α-TGF-β (10 μg/mL). Another AE environment (AE2) was created by adding the following molecules to the AE described above: b-FGF (0.05 μg/mL) and follistatin (0.5 μg/mL).

Overriding the Negative Effects of Aged Systemic Milieu on Muscle Regenerative Potential

Isolation of satellite cells has been performed as explained in the Materials and Methods section. Primary cultures of myogenic progenitor cells were cultured overnight in Opti-MEM with 1% FBS to promote an exit from cell cycle and return to quiescence. After the transient growth factor withdrawal cells were cultured in control or modified adhesion substrates (as indicated) in the presence of Opti-MEM containing 5% of either young or old mouse sera for 60 hours. These progenitor cells were fixed and analyzed by immunofluorescence for their myogenic potential, measured as an ability to form de-novo eMHC⁺ myotubes. As shown in FIG. 5, old serum inhibited myogenic potential of these young progenitor cells cultured with control adhesion substrate, however, local release of Delta and Shh in combination with anti-TGF-β neutralizing antibody from the modified adhesion substrate has overridden the inhibition of myogenic potential imposed by the old serum and has enhanced formation of eMHC⁻ colonies in both young, and importantly, old systemic milieu.

Thus, a productive regenerative behavior of even young progenitor cells is inhibited by the aged systemic milieu and the modified adhesion substrates designed in this work allow overcome such inhibition and restore muscle regenerative potential.

Yet another useful outcome of our work is the potential to develop a better microenvironment for cell transplantation studies. Currently, there is no method that allows successful transplantation of myogenic progenitor cells. In this study, we have defined conditions that could improve the regenerative potential of transplanted cells, by allowing local control of their proliferation and terminal myogenic differentiation. Current experiments, shown in FIG. 4, demonstrate that negative effects of the aged environment can, in fact, be overcome and thus, muscle regenerative potential can be controlled efficiently when myogenic progenitor cells are transplanted in the context of the modified ECM tested in this work. In these current applications, the concentration and specific combinations of growth-promoting and differentiation-promoting factors in the ECM is attenuated to produce maximum myogenic potential in the presence of aged extrinsic milieu (FIG. 4).

It is understood that the examples and embodiments described herein are or illustrative purposes only and that various modifications or changes in light thereof will be suggested to persons skilled in the an and are to be included within the spirit and purview of this application and scope of the appended claims. All publications, patents, and patent applications cited herein are hereby incorporated by reference for all purposes.

What is claimed is:

1. A modified adhesion substrate composition comprising:
an extracellular matrix and at least two anti-aging factors, wherein said composition provides an anti-aging environment for a cell.

2. The modified adhesion substrate composition of claim 1, further comprising a stem cell.

3. The modified adhesion substrate composition of claim 3, wherein said stem cell is a member selected from a satellite cell and a myogenic progenitor cell.

4. The modified adhesion substrate composition of claim 1, further comprising a terminally differentiated cell.

5. The modified adhesion substrate composition of claim 4, wherein said terminally differentiated cell is a myofiber.

6. The modified adhesion substrate composition of claim 1, wherein the anti-aging factors are members selected from DLL4, Shh, α-TGF-β, b-FGF and follistatin.

7. The modified adhesion substrate composition of claim 6, wherein the composition comprises at least three anti-aging factors.

8. The modified adhesion substrate composition of claim 7, wherein said anti-aging factors are DLL4, Shh and α-TGF-β.

9. The modified adhesion substrate composition of claim 8, wherein said DLL4 is present in said composition in a concentration of from about 0.1 μg/mL to about 1 μg/mL, Shh is present in said composition in a concentration of from about 0.1 μg/mL to about 1 μg/mL and α-TGF-β is present in said composition in a concentration of from about 1 μg/mL to about 50 μg/mL.

10. The modified adhesion substrate composition of claim 9, wherein said DLL4 is present in said composition in a concentration of about 0.5 μg/mL, Shh is present in said composition in a concentration of about 0.5 μg/mL and α-TGF-β is present in said composition in a concentration of about 10 μg/mL.

11. The modified adhesion substrate composition of claim 1, wherein the composition comprises at least five anti-aging factors.

12. The modified adhesion substrate composition of claim 11, wherein said anti-aging factors are DLL4, Shh, α-TGF-β, b-TGF and follistatin.

13. The modified adhesion substrate composition of claim 12, wherein said DLL4 is present in said composition in a concentration of from about 0.1 μg/mL to about 1 μg/mL, Shh is present in said composition in a concentration of from about 0.1 μg/mL to about 1 μg/mL, α-TGF-β is present in said composition in a concentration of from about 1 μg/mL to about 50 μg/mL, b-FGF is present in said composition in a concentration of from about 0.01 μg/mL to about 0.1 μg/mL and follistatin is present in said composition in a concentration of from about 1 μg/mL to about 10 μg/mL.

14. The modified adhesion substrate composition of claim 13, wherein said DLL4 is present in said composition in a concentration of about 0.5 μg/mL, Shh is present in said composition in a concentration of about 0.5 μg/mL, α-TGF-β is present in said composition in a concentration of about 10 μg/mL, b-FGF is present in said composition in a concentration of about 0.05 μg/mL and follistatin is present in said composition in a concentration of about 0.5 μg/mL.

15. An modified adhesion substrate composition comprising:
an extracellular matrix and at least two growth factors, wherein said composition provides a growth environment for a cell.

16. The modified adhesion substrate composition of claim 15, further comprising a stem cell.

17. The modified adhesion substrate composition of claim 16, wherein said stem cell is a member selected from a satellite cell and a myogenic progenitor cell.

18. The modified adhesion substrate composition of claim 15, wherein said growth factors are members selected from basic-FGF, follistatin and α-TGF-β.

19. The modified adhesion substrate composition of claim 15, wherein the composition comprises at least three anti-aging factors, and said factors are basic-FGF, follistatin and α-TGF-β.

20. The modified adhesion substrate composition of claim 19, wherein said basic-FGF is present in said composition in a concentration of from about 0.01 μg/mL to about 0.1 μg/mL, follistatin is present in said composition in a concentration of from about 0.1 μg/mL to about 1 μg/mL and α-TGF-β is present in said composition in a concentration of from about 1 μg/mL to about 50 μg/mL.

21. The modified adhesion substrate composition of claim 20, wherein said basic-FGF is present in said composition in a concentration of about 0.5 μg/mL, follistatin is present in said composition in a concentration of about 0.5 μg/mL and α-TGF-β is present in said composition in a concentration of about 10 μg/mL.

22. The modified adhesion substrate composition of claim 15, further comprising differentiation media.

23. An modified adhesion substrate composition comprising:
an extracellular matrix and at least two differentiation factors, wherein said composition provides a differentiation environment for a cell.

24. The modified adhesion substrate composition of claim 23, further comprising a stem cell.

25. The modified adhesion substrate composition of claim 25, wherein said stem cell is a member selected from a satellite cell and a myogenic progenitor cell.

26. The modified adhesion substrate composition of claim 23, further comprising a terminally differentiated cell.

27. The modified adhesion substrate composition of claim 26, wherein said terminally differentiated cell is a myofiber.

28. The modified adhesion substrate composition of claim 23, wherein said differentiation factors are members selected from myostatin, IGF-1 and TGF-β.

29. The modified adhesion substrate composition of claim 28, wherein the composition comprises at least three differentiation factors and said factors are myostatin, IGF-1 and TGF-β.

30. The modified adhesion substrate composition of claim 29, wherein said myostatin is present in said composition in a concentration of from about 0.01 μg/mL to about 1 μg/mL, IGF-1 is present in said composition in a concentration of from about 0.1 μg/mL to about 1 μg/mL and TGF-β is present in said composition in a concentration of from about 0.01 μg/mL to about 0.1 μg/mL.

31. The modified adhesion substrate composition of claim 30, wherein said myostatin is present in said composition in a concentration of about 0.1 μg/mL, IGF-1 is present in said composition in a concentration of about 0.5 μg/mL and TGF-β is present in said composition in a concentration of about 0.02 μg/mL.

32. The modified adhesion substrate composition of claim 23, further comprising growth media.

33. A method of rejuvenating stem cells in a patient, comprising
(a) contacting a stem cell with the composition of claim 1 with said stem cell,
thereby rejuvenating said stem cell.

34. A method of transplanting stem cells into a patient, comprising
(a) administering the composition of claim 2 to said patient,
thereby transplanting said stem cell into said patient.

35. A method of treating muscle injury in a patient, said method comprising:
(a) administering the composition of claim 3 to said patient,
thereby treating said muscle injury.
